(19) United States
(12) Patent Application Publication
(10) Pub. No.: US 2022/0261870 A1
(43) Pub. Date: Aug. 18, 2022

(54) SYSTEM AND METHOD FOR PROVIDING DRUG PRICING INFORMATION

(71) Applicant: David A. Harrell, Naples, FL (US)

(72) Inventor: David A. Harrell, Naples, FL (US)

(21) Appl. No.: 17/174,538

(22) Filed: Feb. 12, 2021

(51) Int. Cl. G06Q 30/06; G16H 40/20; G16H 20/10; G06Q 30/02; G06Q 10/10; G06Q 40/08

(52) U.S. Cl. CPC G06Q 30/0629; G16H 40/20; G16H 20/10; G06Q 40/08; G06Q 30/0283; G06Q 10/10; G06Q 30/0639

(57) ABSTRACT

A system and method for determining a lowest payment method and/or combination of payment methods for purchasing one or more drug prescriptions wherein a lowest out-of-pocket expense to a patient is determined based on various pricing and reimbursement variables.

### FIELD OF THE INVENTION

This invention relates to methods for searching and comparing prices for pharmaceutical prescriptions, and more particularly, a system and method for determining a lowest payment method and/or combination of payment methods for purchasing one or more drug prescriptions wherein a lowest out-of-pocket expense to a patient is determined based on various pricing and reimbursement variables.

### BACKGROUND OF THE INVENTION

Conventional methods for gathering and reporting drug prices operate by obtaining drug pricing information from multiple pharmacy benefit managers (“PBM”), which is then simply passed on to patients looking for a lowest offered price. For example, a conventional system may obtain, calculate, and/or estimate drug prices that are available under contracts or agreements between PBMs and various pharmacies, which is information typically not available to average consumers.

In response to a request for a price of a specific drug, conventional systems will display relevant prices for each pharmacy chain and/or display prices found in a particular geographic area. The users can compare prices for a particular drug and determine which pharmacy they prefer to purchase the drug from.

Conventional drug pricing methods concentrate on a lowest retail price and, however, the cheapest retail price of a prescription may not equate to the lowest out-of-pocket expense for a patient. Conventional systems fail to take many factors and variables into account that affect the amount a patient pays out-of-pocket at the register. For example, a conventional drug pricing method may find a cheapest price for a prescription but a higher price at another pharmacy may be a better deal for a patient based on the patient's insurance, co-pays, reimbursement sources and other variables. Without additional information and analysis, the patient mistakenly ends up purchasing a prescription for a price they believe is saving the most money, when in reality it is not.

Therefore, a need exists for a system and method for determining a lowest payment method and/or combination of payment methods for purchasing one or more drug prescriptions wherein a lowest out-of-pocket expense to a patient is determined based on various pricing and reimbursement variables.

### SUMMARY OF THE INVENTION

The primary object of the present invention is to provide a system and method for determining a lowest payment method and/or combination of payment methods for purchasing one or more drug prescriptions wherein a lowest out-of-pocket expense to a patient is determined based on various pricing and reimbursement variables.

The present invention fulfills the above and other objects by providing a system for determining the best purchase method wherein a combination of variables are examined, such as cash, insurance reimbursement, purchasing a generic version vs. a name brand version, quantity of the prescription, pharmacy benefit mangers' (PBM) pricing information, manufacturers' coupons, location of patient and so forth, and to determine how each variable works together or independently to offset a price of a desired drug for a patient. By accessing data through private and public databases, the system and method of the present invention is able to compare and contrast a plurality of variables to determine a best out of pocket scenario for a patient.

Another element of the present invention comprises an interactive reminder system for patients to confirm medicine has been administered per each dose. If a patient fails to take a dose, then one or more actions may be taken depending on the reason for not taking the dose. These actions may include sending a request to a pharmacy to re-fill the prescription, contacting the patient's physician or caretaker, sending information about the importance of taking your medications as prescribed, sending updates and/or alerts regarding the drug, and so forth.

An additional element of the present invention also compromises a reminder system for patients to refill their medications. This includes the inclusion of the users' specific medication, dose, and prescription timelines. Reminders preferably include a coupon for a pharmacy refilling the prescription confirming the purchase method providing the lowest-out-of-pocket purchase method and/or a coupon for a new pharmacy if a lower purchase method is determined at a new pharmacy prior to a date for re-filling the prescription.

The above and other objects, features and advantages of the present invention should become even more readily apparent to those skilled in the art upon a reading of the following detailed description in conjunction with the drawings wherein there is shown and described illustrative embodiments of the invention.

### BRIEF DESCRIPTION OF THE DRAWINGS

In the following detailed description, reference will be made to the attached drawings in which:

FIG. 1 is a schematic diagram showing an online environment in which the system and method of the present invention operates;

FIG. 2 is a flow chart showing steps for carrying out the system and method of the present invention wherein steps for determining a lowest out of pocket cost for a patient are illustrated; and

FIG. 3 is a flow chart showing steps for carrying out the system and method of the present invention wherein steps for monitoring a user's user of a specific drug are illustrated.

### DESCRIPTION OF THE PREFERRED EMBODIMENTS

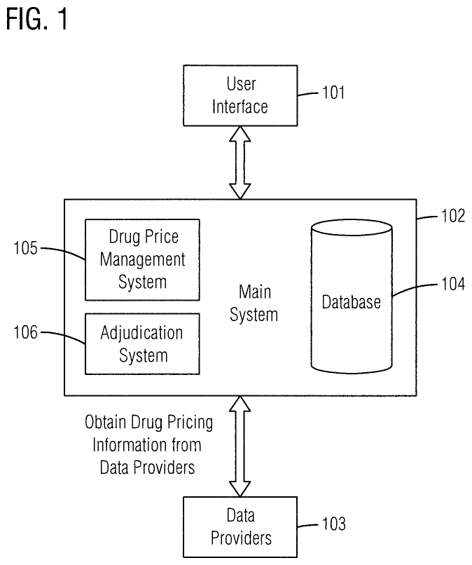

With reference to FIG. 1, an example data flow diagram for providing drug pricing information from multiple data providers to a user interface is illustrated. The method and system of the present invention is accessed online via a user interface 101 that is presented on an online site accessed using an electronic device capable of accessing a software application of the present invention through a provider network 102 having online access to a plurality of data providers 103.

A user, such as a patient or retail purchaser of a drug, may access the provider network 102 over the Internet or app and user interface presented on the user's electronic device, which may be any suitable computing device, such as a smart phone, a tablet, a laptop, and so forth. The provider network 102 comprises at least one database 104, connected to a drug price management system 105 or module and an adjudication system 106 or module.

The information obtained, analyzed and stored within the provider network 102 is obtained from third party data providers 103, which may comprise drug manufacturers, formulary data, PBMs, pharmacies, health insurance providers, government entities and so forth. The information obtained, analyzed and stored within the provider network 102 comprises pricing variables and reimbursement variables provided by the data providers 103. Pricing and reimbursement variables may comprise variables relating to PBM data, geographic location, cash, insurance reimbursement, purchasing a generic version vs. a name brand version, quantity of the prescription, manufacturers' coupons, location of patient and so forth.

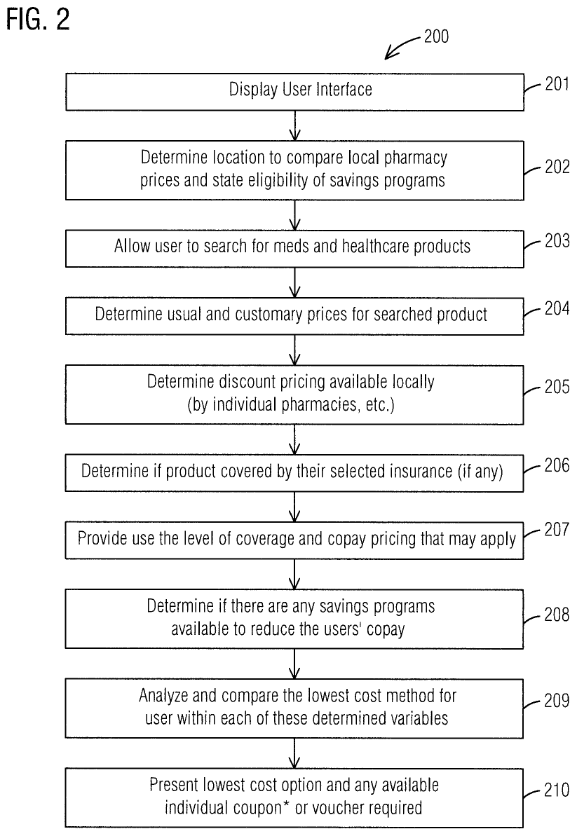

With reference to FIG. 2, a flow chart showing steps for carrying out the system and method of the present invention wherein steps for determining a lowest out of pocket cost for a patient is illustrated. First, a graphical user interface is presented on a user's electronic device 201 that provides the user with access to an online site capable of accessing a software application of the present invention through a provider network having online access to a plurality of third-party data providers, as illustrated in FIG. 1. A user may be a patient, a parent or guardian of a patient, a caregiver or any individual purchasing a prescription drug from a retail pharmacy. The graphical user interface provides capability for a user to enter information relating to a prescribed drug the user wishes to search after which, a lowest out-of-pocket price for the user based on a plurality of variables provided by the user and third-party data providers. Third party data providers may comprise drug manufacturers, formulary data, PBMs, pharmacies, health insurance providers, government entities and so forth, that provide data relating to pricing and reimbursement variables, may include variables relating to PBM data, geographic location, cash, insurance reimbursement, purchasing a generic version vs. a name brand version, quantity of the prescription, manufacturers' coupons, location of patient and so forth.

Next, a geographical location of the user is determined using location services on the user's electronic device or from the user manually entering his or her location into the graphical user interface 202. Then, the user may search for one or more drugs using the graphical user interface 203. Next, the determined location is used to locate retail pharmacies within a specified distance from the user's determined location and customary prices for a searched drug at the located retail pharmacies are obtained 204. Then, availability of discount pricing is determined at the located pharmacies 205. This may also comprise determining available savings programs in the specified geographic area and eligibility requirements for any savings programs. Next, any insurance coverage for the desired drug is determined 206 and a level of coverage and copay pricing is provided to the user via the graphical interface 207. Then, availability of any savings programs relating to the user's determined co-pay is determined and provided to the user 208. Next, the lowest cost method for the user within each determined variable is determined 209. Finally, the lowest cost method for the user is presented on a coupon or voucher via the graphical interface 210.

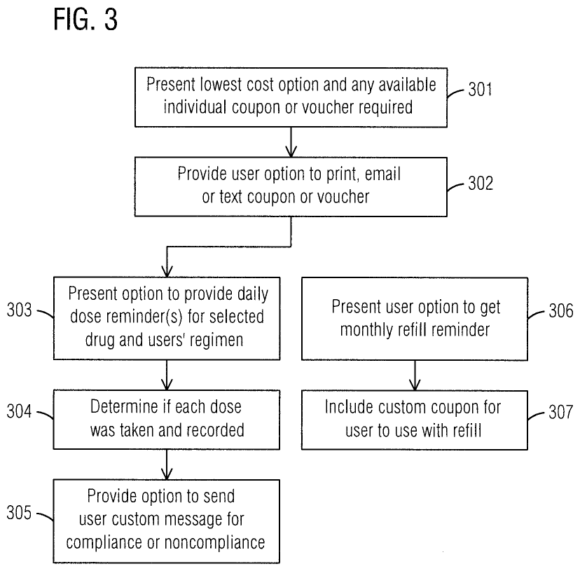

Finally, with reference to FIG. 3, a flow chart showing steps for carrying out the system and method of the present invention wherein steps for monitoring a user's user of a specific drug is illustrated. First, the lowest cost method for the user is presented on a coupon or voucher via the graphical interface 301, as described in FIG. 2. Then, the user is provided with capability to print, email or text the coupon or voucher 302.

Next, the user is provided with capability to select reminders for administering the drug 303 to be delivered via the service provider network illustrated in FIG. 1. The user is then provided with subsequent reminders to take required doses at specified times. Next, the user confirms if he or she took the required dose using the graphical interface 304. Then, the user is sent a message confirming compliance or non-compliance 305.

The user may also be provided with capability to select reminders for refiling the prescription 306. Future reminders are delivered to the user via the service provider network with a coupon or voucher for the drug to be re-filled 307.

Reminders may comprise inclusion of a user's specific medication, dose, and prescription timelines. Reminders preferably include a coupon for a pharmacy refilling the prescription confirming the purchase method and thus, the lowest-out-of-pocket purchase method for the patient and/or a coupon for a new pharmacy if a lower purchase method is determined at a new pharmacy prior to a date for re-filling the prescription.

It is to be understood that while a preferred embodiment of the invention is illustrated, it is not to be limited to the specific form or arrangement of parts herein described and shown. It will be apparent to those skilled in the art that various changes may be made without departing from the scope of the invention and the invention is not to be considered limited to what is shown and described in the specification and drawings.

Having thus described my invention, I claim:

1. A system for determining a lowest out-of-pocket amount available to a specific patient for a prescription drug, said system comprising:
a graphical user interface provided by a service provider network and connected for electronic communication through a networked communications system over the Internet;
said graphical user interface being accessible via a user's electronic device over the Internet;
said service provider network being connected to at least one database for storing information, a management system for managing inquires and data, and an adjudication system for comparing a plurality of third-party data obtained from third-party data providers;
third-party data being selected based on a plurality of search parameters; and
said third-party data being pricing and reimbursement variables that affect a determination of a final lowest out-of-pocket payment option for the user.

2. The system of claim 1 wherein:
said graphical user interface provides capability for a user to enter information relating to a prescribed drug the user wishes to search.

3. The system of claim 1 wherein:
said third party data providers comprise at least one drug manufacturers, at least one retail pharmacy, at least one PBM, and at least one health insurance provider.

4. The system of claim 1 further comprising:
a determination of user's geographical location being determined using the user's electronic device.

5. The system of claim 4 further comprising:
a determination of a plurality of retail pharmacies within a specified distance from the user's determined geographic location; and
a determination of customary prices for a searched drug at the plurality of located retail pharmacies.

6. The system of claim 5 further comprising:
a determination of discount pricing being available at one or more of the is determined at the plurality of located retail pharmacies.

7. The system of claim 6 further comprising:
a determination of available savings programs in a specified geographic area being made.

8. The system of claim 7 further comprising:
a determination of available insurance coverage for the desired drug being made.

9. The system of claim 8 further comprising:
a determination of a lowest cost method for the user being determined; and
the determined lowest cost method for the user being presented via the graphical interface.

10. The system of claim 9 further comprising:
monitoring a user's use of drug via at least one electronic reminder.

11. A method for determining a lowest out-of-pocket amount available to a specific patient for a prescription drug, said method comprising:
providing a graphical user interface from a service provider network and connected for electronic communication through a networked communications system over the Internet;
said graphical user interface being accessible via a user's electronic device over the Internet;
said service provider network being connected to at least one database for storing information, a management system for managing inquires and data, and an adjudication system for comparing a plurality of third-party data obtained from third-party data providers;
selecting third-party data based on a plurality of search parameters; and
said third-party data being pricing and reimbursement variables that affect a determination of a final lowest out-of-pocket payment option for the user.

12. The method of claim 11 wherein:
said graphical user interface provides capability for a user to enter information relating to a prescribed drug the user wishes to search.

13. The method of claim 11 wherein:
said third party data providers comprise at least one drug manufacturers, at least one retail pharmacy, at least one PBM, and at least one health insurance provider.

14. The method of claim 11 further comprising:
determining a user's geographical location using the user's electronic device.

15. The method of claim 14 further comprising:
determining a plurality of retail pharmacies within a specified distance from the user's determined geographic location; and
determining customary prices for a searched drug at the plurality of located retail pharmacies.

16. The method of claim 15 further comprising:
determining discount pricing being available at one or more of the is determined at the plurality of located retail pharmacies.

17. The method of claim 16 further comprising:
determining available savings programs in a specified geographic area being made.

18. The method of claim 17 further comprising:
determining available insurance coverage for the desired drug being made.

19. The method of claim 18 further comprising:
determining a lowest cost method for the user being determined; and
presenting the determined lowest cost method for the user via the graphical interface.

20. The method of claim 19 further comprising:
monitoring a user's use of drug via at least one electronic reminder.
